(19) United States
(12) Patent Application Publication
(10) Pub. No.: US 2013/0090681 A1
(43) Pub. Date: Apr. 11, 2013

(54) DILATING DEVICE

(71) Applicants: Peter Young, King's Lynn (GB); Parvez Moondi, Whissonsett (GB)

(72) Inventors: Peter Young, King's Lynn (GB); Parvez Moondi, Whissonsett (GB)

(73) Assignee: Cook Medical Technologies LLC, Bloomington, IN (US)

(21) Appl. No.: 13/697,591

(22) PCT Filed: May 13, 2011
(86) PCT No.: PCT/US11/36427

§ 371 Date: Dec. 5, 2012

(30) Foreign Application Priority Data
May 15, 2010 (GB) 1008109.9

(51) Int. Cl. A61M 29/00

(52) U.S. Cl. CPC A61M 29/00
USPC 606/198

(57) ABSTRACT

A device for dilating an opening in a chest wall to facilitate insertion of a chest tube into the pleural cavity. A forceps has a blunt distal tip suitable for carrying out blunt dissection of the tissue of the chest wall, and a channel along one arm of the forceps. A wire extends through the channel to provide access to the pleural cavity. Following removal of the forceps from the wire, the distal end of the chest tube can be inserted into the pleural cavity over the wire.

### BACKGROUND

The present invention relates to the field of fluid drainage from a body cavity, and more particularly, to a device for use in drainage of fluid from the pleural cavity of a patient.

in the human body, the lungs are surrounded by the pleura. The pleura is a serous membrane which folds back upon itself to form a two membrane structure. The two membranes are known as the parietal pleura and the visceral pleura, respectively. The parietal (outer) pleura lines the chest wall, while the visceral (inner) pleura surrounds the lung. The space between the two pleurae layers, referred to as the pleural cavity or the pleural space, contains a thin layer of pleural fluid. This fluid enables each plume layer to smoothly slide relative to the other layer during respiration.

Pleural effusion refers to a condition that occurs when an excess of fluid accumulates in the pleural cavity. Typically, such accumulation results from chest, trauma experienced by the patient. The collection of air in the pleural cavity results in a condition commonly referred to as pneumothorax. The collection of blood in the pleural cavity results in a condition commonly referred to as hemothorax. Other fluids that may collect in the pleural cavity include serous fluid (hydrothorax), chyle (chylothorax), and pus (pyothorax).

The presence of excessive amounts of fluids in the pleural cavity impairs the breathing ability of the patient by limiting the ability of the lungs to expand during inhalation. In order to drain excess fluid, a chest drain tube (“chest tube”) may be inserted into the pleural cavity. Inserting a chest tube is a potentially hazardous activity that has been associated with complications, such as damage to the lungs, heart and liver.

There are many techniques used to insert chest tubes into the pleural cavity, most of which broadly fall within the following two techniques or minor variations thereof. The first technique (Technique 1) is the insertion of the chest tube following blunt dissection utilizing, e.g., surgical forceps. Initially, an incision is made parallel to the rib. The subcutaneous layers and the intercostal muscles are dissected by introducing the blunt-tipped forceps into the incision in the closed condition. Once these tissues have been dissected the forceps is opened to spread the parietal pleura and the intercostal muscles. The operator's finger is then inserted into the opening to ensure that there are no underlying structures which could be unintentionally penetrated, and to ensure that sufficient area is present to enable the chest tube to be safely inserted into the pleural cavity. A disadvantage of this technique is that it typically requires that a relatively large hole be created in the chest. Additionally, the technique is sometimes difficult to perform, as the chest tube can be difficult to insert once the operator's finger is removed from the chest cavity. Finally, the operator's finger is also susceptible to injury upon insertion into the opening.

The second technique (Technique 2) is often referred to as the Seldinger technique. This technique requires the insertion of a hollow needle through the chest wall and into the thoracic (pleural) cavity, followed by introduction of a wire through a bore of the needle. The needle is then withdrawn, leaving the distal end of the wire in the pleural cavity. A series of tapered dilators (such as three) are sequentially advanced (small to large) over the wire guide to dilate the tissue of the chest wall, and form an opening of desired size. After removal of the largest dilator, the chest tube, typically with an inserter/obturator, is placed over the wire guide, and the distal end of the tube is directed into the pleural cavity. This technique has the advantage of allowing a smaller hole to be created in the chest wall than the previous technique. It is also a relatively easy technique to perform. However, the procedure can be time consuming, and requires the use of multiple devices for its completion. In addition, the needle tip and pointed dilators/obturators are inserted blindly in the direction of the posterior pleural cavity, which blind insertion presents a risk of puncture of the lung or other body organ if not carried out properly. Even when ultrasound imagery is used to enhance visibility, accuracy depends upon operator skill, as the fluid and body organs can shift between initial examination and operative procedure. Furthermore, when treating conditions such as a pneumothorax, ultrasound may not reliably distinguish between lung and an air-filled pleural space.

It is desired to provide a device useful for insertion of a tubular member, such as a chest tube. In a body cavity that addresses the problems that occur in the prior art.

### BRIEF SUMMARY

The present invention addresses the problems of the prior art, in one form thereof, a dilating device is provided for accessing a body cavity for insertion of a tubular member therein, A pair of elongated members is pivotally joined to each other along a length of each of the elongated members. Each elongated member has a pivot point for joinder to the other elongated member, a proximal end accessible to an operator during use of the dilating device, and a distal end for insertion through tissue adjacent the body cavity. Each distal end terminates at a substantially blunt distal tip. The respective distal ends are movable between a closed position wherein the distal ends are at least substantially contiguous to each other, and an open position wherein the distal ends are separable to dilate the tissue. At least one of the elongated members has a channel extending along a length thereof. The channel has an entry opening, and has an exit opening distal of the entry opening along the length of the elongated member.

In another form thereof, a system is provided for accessing a pleural cavity for insertion of a tubular member therein. A generally elongated member has a proximal end, a distal end extending to a substantially blunt distal tip, and a channel extending along a length of the generally elongated member between the proximal and distal ends. The channel has an entry opening, and has an exit opening distal of the entry opening along the generally elongated member length. The exit opening may be spaced from the entry opening by a distance of at least about 4 cm. The elongated member has sufficient rigidity to enable the introducer member to penetrate body tissue surrounding the pleural cavity. A wire is received in the channel. The wire has a proximal end accessible to an operator through the entry opening, and a distal end extendable through the exit opening into the pleural cavity.

In yet another form thereof, a method is provided for providing access to a body cavity of a patient for insertion of a tubular member. A dilating device is positioned for insertion through a body wall. The dilating device comprises a pair of elongated members pivotally joined to each other along a length of each of the elongated members. Each elongated member has a proximal end accessible to an operator during use of the dilating device, and a distal end for initial insertion through tissue of the body wall. The respective distal ends are movable between a closed position wherein the distal ends are at least substantially contiguous to each other for facilitating blunt dissection of the body wall tissue during an initial insertion of the dilating device, and an open position wherein the distal ends are separable to dilate the tissue. At least one of the elongated members has a channel extending along a length thereof. The channel has an entry opening, and has an exit opening distal of the entry opening along a length of the elongated member. A blunt dissection of the tissue is carried out by advancing the closed distal ends of the dilating device through an incision in the body wall to the body cavity. A wire is arranged through the channel such that a proximal end of the wire extends through the entry opening for accessibility to the operator, and a distal end of the wire extends through the exit opening into the body cavity.

One feature of the present invention is that once the wire is in place, the channeled forceps can be removed over the wire, and then a chest tube can be inserted over the wire, with or without the use of one or more dilators to widen the tract. Modified forceps for introduction of a wire guide have been used in the art. For example, in U.S. Pat. No. 5,279,285, incorporated by reference herein, a surgical forceps for placement of a guidewire in the trachea during a tracheotomy operation is disclosed. This forceps design has two half channels, each half channel within one of the arms of the forceps tip. As a result, this forceps must be closed at all times to form the actual channel that receives the wire. The forceps channel has a very short length, and does not extend beyond the forceps hinge. On the other hand, the channel of the forceps disclosed herein may extend more than 4 cm for placement of a chest tube, and may extend beyond the hinge. Additionally, the wire extends along a channel in a single arm of the forceps. Thus, it does not matter whether the forceps are open or closed at any one time, as the channel remains intact.

Chinese patent document CN 201067418, incorporated by reference herein, discloses a dilating forceps with a wire channel for performing a tracheostomy. When the dilating forceps is released, the forceps automatically restore to their original arrangement. As with the forceps of the '285 patent, these forceps are intended for procedures whereby the wire is initially placed through a needle, e.g., via a Seldinger procedure, and the forceps are thereafter passed over an already positioned guiding wire.

The forceps disclosed herein are utilized in a contrary manner. The forceps are first properly positioned with the distal end in the pleural space, and the wire is thereafter guided through the channel to the required position. The prior art forceps designs have short channels which do not extend beyond the hinge of the forceps, because there is no benefit for a long channel when performing a tracheostomy by placing the forceps channel over a positioned wire. In one form thereof, the present invention utilizes a longer channel, since the proximal entry hole of the channel needs to be accessible to the operator with the forceps in the correct anatomical position. The present invention enables the forceps to be inserted in either an open or a closed position, and the forceps may be inserted such that the hinge is positioned within the layer(s) of tissue.

### BRIEF DESCRIPTION OF THE DRAWINGS

FIG. 1 is a perspective view of a dilating device according to one embodiment of the present invention;

FIG. 2 is a perspective view of a dilating device according to another embodiment of the present invention;

FIG. 3 is a side view of a dilating device according to yet another embodiment of the present invention;

FIG. 4 is a sectional view of the dilating device of FIG. 3 taken along line A-A;

FIG. 5 illustrates the insertion of the distal tip of a chest tube into the pleural space over the wire; and

FIG. 6 illustrates the position of the chest tube following withdrawal of the wire.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated apparatus, and such further applications of the principles of the invention as Illustrated therein being contemplated as would normally occur to one skilled in the art to which the invention relates.

In the following discussion, the terms “proximal” and “distal” will be used to describe the opposing axial ends of a dilating device for use in the insertion of a tubular member into a body cavity of a patient, as well as opposing axial ends of component features. The term “proximal” is used in its conventional sense to refer to the end of the dilating device, or component feature, that is closest to the operator during use. The term “distal” is used in its conventional sense to refer to the end of the dilating device, or component feature, that is initially inserted into the patient, or that is closest to the patient during use. In one preferred use, the dilating device may be used for insertion of a distal end of a chest tube into the pleural cavity of a patient. Those skilled in the art will recognize that the dilating device may also be used for insertion of other tubular members in other body cavities of the patient. Although minor modification of the preferred embodiments described herein may be required for such uses, such modification is believed to only involve routine change for those skilled in the art.

The inventive dilating device is suitable for use for insertion of a tubular member. As stated above, prior art techniques for insertion of a tubular member, such as a chest tube, into a body cavity, such as the pleural cavity, typically involved blunt dissection (Technique 1) or insertion of a wire via the Seldinger technique (Technique 2). Each of these techniques is associated with certain disadvantages, many of which have been described herein. The inventive dilating device allows insertion of a distal end of a tubular member into a body cavity in a manner that minimizes these, and other, disadvantages associated with prior art techniques.

In one form, a forceps is used for introduction into the pleural cavity. The forceps may be initially introduced by blunt dissection. This known technique may include a finger sweep within the thorax to determine whether any underlying structures that may cause interference with the insertion are present, however a finger sweep is not required. One general manner of introduction via this technique is described in U.S. Pat. No. 4,608,982, incorporated by reference herein.

As indicated by the '982 patent, it is well known in the medical arts to use a blunt-tipped forceps for dissection. However, the known technique typically requires the creation of a relatively large hole in the chest. This technique can be difficult to perform properly, and may result in difficulties in inserting the chest tube following removal of the operator's finger from the chest cavity. The other prior art (Seldinger) technique described above does not require that the operator insertion his/her finger into the chest cavity as described, but rather, involves the insertion of the distal end of a wire into the pleural cavity. However, this technique necessitates the insertion of a needle and successive pointed dilators/obturators in the direction of the posterior pleural cavity as discussed, and therefore, presents a risk of puncture of the lung or other organ if not carried out properly.

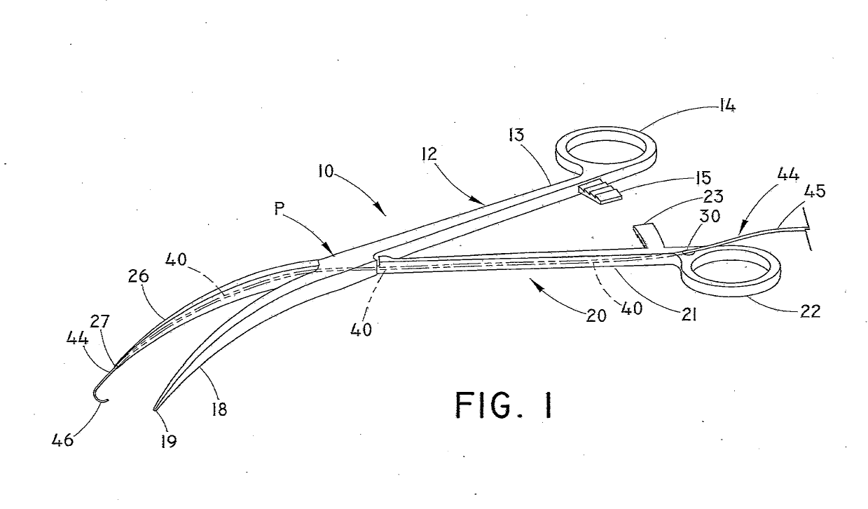

The modified forceps as described herein enables the operator to incorporate favorable features from each of the prior art techniques, while at the same time minimizing the disadvantages. One form of such modified forceps is illustrated in FIG. 1. When utilizing the forceps as described, blunt dissection can be carried out, thereby eliminating needle trauma and the other difficulties encountered when carrying out the Seldinger technique. At the same time wire entry can be made into the pleural cavity through a channel within the forceps, without the necessity of a finger sweep.

FIG. 1 illustrates one form of a forceps 10 according to an embodiment of the present invention. Forceps 10 includes a pair of elongated members 12, 20 pivotally engaged along a length “P” of the forceps in any well-known fashion, such as via a hinge (not shown). Elongated members 12, 20 include respective proximal ends 13, 21, and distal ends 18, 26. The proximal end 13, 21, of elongated members 12, 20 can include finger loops 14, 22 to facilitate grasping of the forceps by the operator. If desired, the proximal end of elongated members 12, 20 can also include optional cooperating members 15, 23. Cooperating members 15, 23 can be provided, e.g., as spacers to maintain a distance between elongated members 12, 20, or as locking members to enable elongated members 12, 20 to be locked together for, e.g., maintaining the distal ends 18, 26 in a closed or otherwise fixed position. Preferably, distal tips 18, 26 have a downward curve along their length. Providing a curved distal region may enhance the ability of the operator to reach the desired portions of the tissue during dissection. It is also preferred that distal tips 18, 26 terminate in a rounded, or blunt, distal tip 19, 27 to minimize the possibility of an inadvertent puncture of the lung or other organs encountered upon entry.

As shown in phantom in FIG. 1, a channel 40 extends interiorly of elongated member 20 along a length of the elongated member. In this embodiment, channel 40 extends from entry opening 30 in proximal portion 21, and exits through an opening at distal tip 27. Channel 40 can be formed through elongated member 20 in any well-known manner, such as by machining, molding, etc. Channel 40 is sized to receive a wire 44 therethrough. Wire 44 is sized such that a proximal wire end 45 extends outwardly of opening 30, and a distal wire end 46 extends through the opening at distal tip 27.

The following discussion describes use of forceps 10 for placement of a chest tube in the pleural cavity of a patient. As stated above, forceps 10 may also be used for insertion of tubular members in other body cavities. Initially, a small incision may be made, e.g., via a scalpel, through the skin and tissue at an appropriate site. If desired, the intercostal space may then be finger palpated as shown, e.g., in the incorporated-by-reference U.S. Pat. No. 4,608,982. Whether or not palpation is carried out, the forceps are arranged such that the respective distal ends 18, 26 are aligned for blunt dissection. Distal tips 19, 27 are inserted into the opening formed in the tissue, and blunt dissection of the opening is carried out in conventional fashion such that distal tips 19, 27 extend through the Intercostal space and muscle layers into the pleural cavity. Although distal ends 18, 26 may be inserted in a closed, or semi-closed condition, this is not required. Rather, in some instances the operator may elect to carry out dissection with the distal ends in a more open condition. Thus, those skilled in the art will appreciate that either alternative is permissible with the disclosed forceps. Since the wire will extend along channel 44 interiorly of the elongated member, the positioning of the wire is not affected by the degree of closure of the elongated members (arms) of the forceps. The blunt tips 19, 27 are less likely to cause injury to underlying structures (e.g., lung, heart, liver) than the sharp needle and/or the dilators/obturators utilized in some conventional techniques.

Following insertion of blunt tips 19, 27 into the pleural cavity, wire 44 is inserted into opening 30, and thereafter threaded through channel 40 of elongated member 20 until the distal end 46 of wire 44 exits tip 27 (FIG. 1) in the pleural cavity. The wire can be directionally guided in the cavity to a preferred location by rotation and/or manipulation of the forceps if desired. Once wire distal end 46 is in place in the cavity, forceps 10 can be withdrawn from the wire. Prior to insertion of the chest tube over the wire, one or more dilators can be used to widen the tract in conventional fashion, if desired.

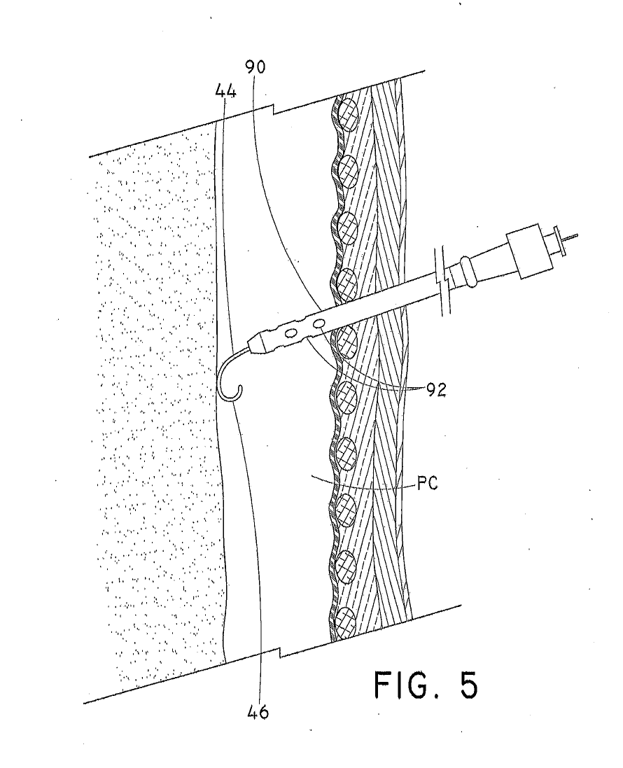

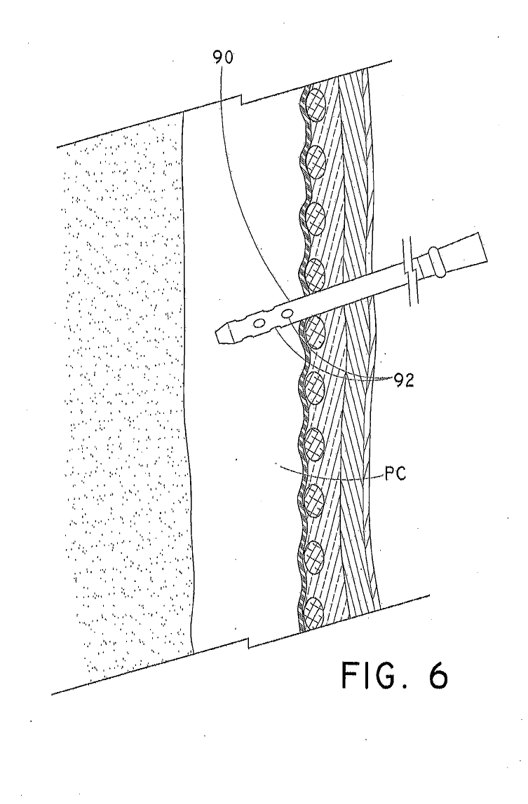

As shown in FIG. 5, a conventional chest tube 90 can then be inserted over the wire 44, such that the side ports 92 of the chest tube extend into the pleural cavity (PC) in well-known fashion. Once the chest tube is properly seated, the wire may be withdrawn, as shown in FIG. 6. The chest tube may then be secured in typical fashion, e.g., by suturing to the skin.

Although FIG. 1 illustrates one preferred embodiment of forceps 10, many variations are also within the scope of the invention. For example, although channel 40 is shown in elongated member 20 in the variation of forceps 10 shown in FIG. 1, this is not required. Rather, the channel can alternatively be provided in elongated member 12 instead of elongated member 20. As a further variation, each elongated member 12, 20 may be provided with a channel 40. As a still further variation, either or both elongated members can be provided with more than one channel.

As a still further variation, channel 40 need not necessarily extend all, or substantially all, of the length of the elongated member as shown in FIG. 1. Typically, however, for drainage of the pleural cavity the channel will extend at least about 4 cm, as it should extend at least the width of the tissue between the exterior of the skin and the cavity. When the forceps are used for insertion of a tubular member in a different body cavity, the length of the channel can be modified accordingly so that the cavity extends at least the length of the tissue between the exterior of the skin and the body cavity of interest. Additionally, the channel need not necessarily span each side of pivot point P as shown, and instead, can be entirely on one axial side, or the other side of the pivot point. For ease of use in accessing the pleural cavity, however, it is preferred to have a channel substantially as shown in FIG. 1. In this regard, the entry opening 30 should be readily accessible to the operator when the forceps are in a proper anatomical position.

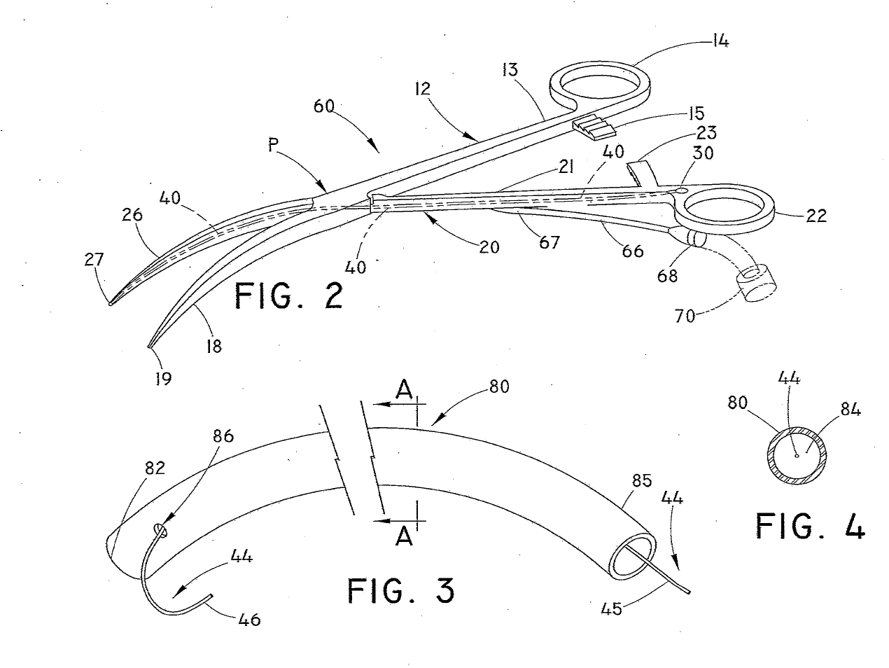

Another embodiment of a forceps 60 is illustrated in FIG. 2. Features in common with forceps 10 are provided with the same reference numeral as in FIG. 1. Forceps 60 includes a connecting element 66. Connecting element 66 includes a first end 67 that fluidly communicates with channel 40, and a second end 68. In the non-limiting embodiment shown in FIG. 2, second end 68 is attached to finger loop 22, although those skilled in the art will appreciate that second end 68 can be arranged at alternative positions, directly engaged with, or not engaged with, forceps 60. Connecting element 66 comprises a conduit in fluid communication with channel 40 for, e.g., the aspiration or other removal of fluid from the pleural cavity via tip 27 and channel 40. To facilitate removal, a syringe, pump, or other suitable apparatus 70 (collectively referred to as “apparatus”, and shown schematically in FIG. 2) may be engaged with connecting element second end 68 by any conventional means. Such engagement may be by temporary means, e.g., a threaded connection, luer connection, friction connection, etc., or by permanent affixation. As a still further alternative, apparatus 70 may be provided as an integral feature of forceps 60 and/or connecting element 66.

Alternatively, connecting element 66 can be provided for introducing a fluid, e.g., a therapeutically useful fluid or a contrast fluid, through channel 40 and tip 27 into the cavity or other body opening. In this instance, apparatus 70 may be a reservoir, syringe, pump, etc., for facilitating introduction of the fluid, which element may be engaged with the connecting element as described above. Those skilled in the art can readily fashion variations of apparatus 70 and connecting elements 66 suitable for use herein.

As a still further variation, apparatus 70 need not be provided to promote fluid introduction or removal but rather, can comprise a mechanism to enhance visualization in the tissue and/or the cavity. In this variation, apparatus 70 may comprise, e.g., a fiber-optic viewing means, a camera, or another viewing or imaging mechanism known to those skilled in the art. Such element need not necessarily be engaged with channel 40 as illustrated herein, as those skilled in the art can readily fashion variations of this arrangement suitable for the intended purpose.

FIGS. 3 and 4 illustrate another embodiment. In this case, instead of providing forceps, such as forceps 10 and 60, for insertion into an opening through the tissue surrounding the cavity, an introducer 80 is provided for such insertion. Introducers are well known in the medical arts and may comprise a sheath formed of a polymer, such as PTFE, or other suitable composition. Alternatively, the introducer may comprise a multi-layer sheath, such as the sheath disclosed in U.S. Pat. No. 5,380,304, incorporated by reference herein, which sheath may include a reinforcing member, such as a coil or a braid, to facilitate introduction into the pleural cavity. If desired, the introducer may be provided with a more flexible distal end and a more rigid proximal end, as well known in the art. The introducer should have sufficient rigidity to enable it to dissect the tissue such that the distal end of the dilator can extend into the pleural cavity.

Preferably, the introducer 80 has a blunt, closed distal end 82 that extends into the cavity. A channel 84 extends through a length of the introducer. In the embodiment shown, wire 44 extends through channel 84 of the introducer. As with the previous embodiments, distal wire end 46 protrudes through an opening 86 at or near the blunt distal end 82 of the introducer. The proximal end 45 of the wire may extend through proximal end 85 of the introducer, or alternatively, may enter channel 84 through an entry at another suitable position along the length of the introducer. If desired, additional channels, or lumens, may be provided along a length of the introducer in well-known fashion.

Any undisclosed details of the construction or composition of the various elements of the disclosed embodiments of the present invention are not believed to be critical to the achievement, of the advantages of the present invention, so long as the elements possess the strength or ability needed for them to perform as disclosed. The selection of these and other details of construction are believed, to be well within the ability of one of ordinary skills in this area, in view of the present disclosure.

It is to be understood, however, that the above-described device and method are merely intended to represent illustrative embodiments of the principles of this invention, and that other devices and methods may be devised by those skilled in the art, without departing from the spirit and scope of the invention.

1. A dilating device for providing access through body tissue to a body cavity for insertion of a tubular member therein, comprising:
a pair of elongated members pivotally joined to each other along a length of each of said elongated members, each elongated member having a pivot point for joinder to the other elongated member, a proximal end accessible to an operator during use of said dilating device, and a distal end for insertion through said tissue, each distal end terminating at a substantially blunt distal tip, said respective distal ends movable between a closed position wherein said distal ends are at least substantially contiguous to each other, and an open position wherein said distal ends are separable for dilating said tissue, at least one of said elongated members having a channel extending through a length thereof, said channel having an entry opening, and having an exit opening distal of said entry opening along the length of said at least one elongated member.

2. The dilating device of claim 1, wherein said exit opening is distal of said pivot point along said length of said at least one elongated member.

3. The dilating device of claim 2, wherein said entry opening is proximal of said pivot point along said length.

4. The dilating device of claim 2, wherein said exit opening is at said distal tip of said elongated member.

5. The dilating device of claim 1, wherein each of said elongated members has a channel extending along a length of said elongated member.

6. The dilating device of claim 5, wherein each said channel has an exit opening distal of said pivot point.

7. The dilating device of claim 2, wherein the channel has a length of at least 4 cm along the length of the elongated member.

8. The dilating device of claim 1, wherein each of said elongated members comprises a finger loop at said proximal end.

9. The dilating device of claim 1, wherein said distal end of each of said elongated members is curved relative to an axis of said proximal end.

10. The dilating device of claim 1, further comprising a wire received in said channel, said wire having a proximal end accessible to said operator through said entry opening, and having a distal end extending through said exit opening.

11. A system for providing access to a body cavity for insertion of a tubular member therein, comprising:
a generally elongated member having a proximal end, a distal end extending to a substantially blunt distal tip, and a channel extending along a length of said generally elongated member between said proximal end and said distal end, said channel having an entry opening, and having en exit opening distal of said entry opening along said generally elongated member length, said elongated member having sufficient rigidity to enable the introducer member to penetrate body tissue surrounding said body cavity; and
a wire received in said channel, said wire having a proximal end accessible to an operator through said entry opening, and having a distal end extendable through said exit opening into said body cavity.

12. The system of claim 11, wherein said body cavity comprises the pleural cavity, and wherein said exit opening is spaced from said entry opening by a distance of at least 4 cm, said system further comprising a chest tube receivable over said wire for draining fluid from said pleural cavity.

13. The system of claim 12, wherein the generally elongated member comprises an introducer.

14. The system of claim 12, further comprising a second generally elongated member, said elongate members pivotally joined to each other along a respective length thereof, each elongated member having a pivot point for joinder to the other elongated member, a proximal end accessible to an operator during use of said introducer, and a distal end for insertion through said tissue, said respective distal ends movable between a closed position wherein said distal ends are at least substantially contiguous to each other, and an open position wherein said distal ends are separable to dilate said tissue, at least one of said elongated members having a channel extending along a length thereof, said channel having an entry opening, and having an exit opening distal of said entry opening along a length of said at least one elongated member.

15. The system of claim 12, further comprising a viewing mechanism associated with the generally elongated member in a manner to provide an operator with a view of said body tissue.

16. The system of claim 12, further comprising a connecting element in fluid communication with an interior of said channel.

17. The system of claim 16, further comprising an apparatus engaged with said connecting element for fluid flow.

18. A method of providing access to a body cavity of a patient for insertion of a tubular member, comprising the steps of:
positioning a dilating device for insertion through a body wall, the dilating device comprising a pair of elongated members pivotally joined to each other along a length of each of said elongated members, each elongated member having a proximal end accessible to an operator during use of said dilating device, and a distal end for initial insertion through tissue of said body wall, said respective distal ends movable between a closed position wherein said distal ends are at least substantially contiguous to each other, and an open position wherein said distal ends are separable for dilating said tissue, at least one of said elongated members having a channel extending along a length thereof, said channel having an entry opening, and having an exit opening distal of said entry opening along the length of said at least one elongated member;
carrying out a blunt dissection of said tissue by advancing the distal ends of the dilating device through an incision in said body wall to the body cavity; and
arranging a wire through the channel such that a proximal end of the wire extends through the entry opening for accessibility to the operator, and a distal end of the wire extends through the exit opening into the body cavity.

19. The method of claim 18, further comprising the steps of:
withdrawing the dilating device over the wire; and
inserting the tubular member over the wire such that a distal end of the tubular member extends into the body cavity.

20. The method of claim 19, wherein the body wall comprises the chest wall, and the tubular member comprises a chest tube, and wherein said chest tube distal end extends into the pleural cavity.
